(19) United States
(12) Patent Application Publication
(10) Pub. No.: US 2017/0290528 A1
(43) Pub. Date: Oct. 12, 2017

(54) SLEEP STUDY USING AN IMPLANTED MEDICAL DEVICE

(71) Applicant: Cardiac Pacemakers, Inc., St. Paul, MN (US)

(72) Inventors: David J. Ternes, Roseville, MN (US); Stephen B. Ruble, Lino Lakes, MN (US); Amy Jean Brisben, St. Paul, MN (US); John D. Hatlestad, Maplewood, MN (US); Keith R. Maile, New Brighton, MN (US); Pramodsingh H. Thakur, Woodbury, MN (US)

(21) Appl. No.: 15/484,982

(22) Filed: Apr. 11, 2017

Related U.S. Application Data
(60) Provisional application No. 62/321,488, filed on Apr. 12, 2016.

(51) Int. Cl. A61B 5/08; A61B 5/00; A61B 5/11; A61B 5/024; A61B 5/0215

(52) U.S. Cl. CPC A61B 5/0826; A61B 5/024; A61B 5/0215; A61B 5/1116; A61B 5/0022

(57) ABSTRACT

A system for performing a sleep study includes an implant able medical device (IMD) having a first sensor configured to obtain a first physiological parameter signal; a second sensor configured to obtain a second physiological parameter signal; and at least one processing device. The at least one processing device is configured to: receive the first physiological parameter signal and the second physiological parameter signal; and identify, based on a set of episode data, an occurrence of an episode during a sleep session, the episode corresponding to a sleep disorder, the set of episode data based on at least one of the first physiological parameter signal and the second physiological parameter signal. The at least one processing device is further configured to generate, based on the first physiological parameter signal and the second physiological parameter signal, a study report, the study report comprising an indication of the episode.

### CROSS-REFERENCE TO RELATED APPLICATION

This application claims priority to Provisional Application No. 62/321,488, filed Apr. 12, 2016, which is herein incorporated by reference in its entirety.

### TECHNICAL FIELD

The present disclosure relates to performing sleep studies by utilizing implant able medical devices.

### BACKGROUND

Patients suffering from sleep disorders may visit a sleep study clinic to analyze and prescribe solutions to sleep disorders. A patient may spend one or more nights at a sleep study clinic so that their sleep patterns and parameters can be observed. Such observation involves sleeping in an atypical environment and being connected to a range of external sensing devices that limit a patient's movement and therefore the ability to sleep as one would normally. A sleep study clinic will also have a different layout than a patient's home, resulting in sleep observations that may not show patients' typical sleep sessions or disturbances involving trips for water or bathroom. Alternatively, patients may resort to using home sleep kits that have a limited range of the types of sleep disorders that can be determined and analyzed and may also include external sensing devices that limit a patient's movement. Moreover, external sensing devices may suffer from noise due to poor connections, perhaps caused by a patient's movement.

### SUMMARY

In an Example 1, a system for performing a sleep study, comprising: an implant able medical device (IMD) configured to be implanted within a patient's body, the IMD comprising a first sensor configured to obtain a first physiological parameter signal; a second sensor configured to obtain a second physiological parameter signal; and at least one processing device configured to: receive the first physiological parameter signal and the second physiological parameter signal; identify, based on a set of episode data, an occurrence of an episode during a sleep session, the episode corresponding to a sleep disorder, the set of episode data based on at least one of the first physiological parameter signal and the second physiological parameter signal; and generate, based on the first physiological parameter signal and the second physiological parameter signal, a study report, the study report comprising an indication of the episode.

In an Example 2, the system of Example 1, wherein the at least one processing device is further configured to identify, at a first time, an onset of the sleep session based on at least one of the first physiological parameter signal and the second physiological parameter signal.

In an Example 3, the system of Example 2, wherein the at least one processing device is further configured to identify, at a second time, an interruption of the sleep session based on at least one of the first physiological parameter signal and the second physiological parameter signal.

In an Example 4, the system of any of Examples 1-4, wherein the first sensor comprises at least one of a respiration sensor, a heart rate sensor, an activity sensor, and a posture sensor.

In an Example 5, the system of any of Examples 1-4, wherein the second sensor comprises at least one of a cardiac sensor, an activity sensor, a posture sensor, a heart sound sensor, an intrathoracic pressure sensor, and a blood pressure sensor.

In an Example 6, the system of any of Examples 1-5, wherein the episode comprises at least one of a breathing cessation episode, an apnea episode, a hypopnea episode, and a restlessness episode.

In an Example 7, the system of either of Examples 1 or 2, the study report comprising correlation information associated with the first physiological parameter signal and the second physiological parameter signal.

In an Example 8, the system of Example 7, wherein the correlation information comprises a correlation between a set of posture information and at least one of a set of apnea information, a set of restlessness information, a set of cardiac information, a set of heart sounds information, a set of blood pressure information, and a set of intrathoracic pressure information.

In an Example 9, the system of any of Examples 1-7, wherein the at least one processing device is further configured to diagnose the sleep disorder based on a count of occurrences of the episode exceeding a threshold.

In an Example 10, a method of performing a sleep study associated with a subject, the method comprising: obtaining, using an implant able medical device (IMD), a first physiological parameter signal; obtaining a second physiological parameter signal; identifying, based on a set of episode data, an occurrence of an episode during a sleep session, the episode corresponding to a sleep disorder, the set of episode data based on at least one of the first physiological parameter signal and the second physiological parameter signal; and generating, based on the first physiological parameter signal and the second physiological parameter signal, a study report, the study report comprising an indication of the episode.

In an Example 11, the method of Example 10, further comprising: identifying, at a first time, an onset of the sleep session based on at least one of the first physiological parameter signal and the second physiological parameter signal; and identifying, at a second time, an interruption of the sleep session based on at least one of the first physiological parameter signal and the second physiological parameter signal.

In an Example 12, the method of either of Examples 10 and 11, wherein generating the study report comprises: generating at least one waveform corresponding to the sleep session, the at least one waveform comprising at least one of a respiration waveform, an electrogram, a pressure waveform, a sound waveform, and an activity waveform; and annotating the at least one waveform to indicate the occurrence of the episode.

In an Example 13, the method of any of Examples 10-12, wherein generating the study report comprises: identifying a plurality of occurrences of additional episodes, each of the plurality of occurrences of additional episodes comprising at least one of a breathing cessation episode, an apnea episode, a hypopnea episode, and a restlessness episode; determining a plurality of postures of the subject during the sleep session; correlating each of the plurality of occurrences of additional episodes with one of the plurality of postures of the subject; and generating correlation information associated with the correlated occurrences of additional episodes and postures.

In an Example 14, the method of Example 13, wherein the plurality of postures comprise at least one of a sitting position, a back-recumbent position, a front-recumbent position, a left-recumbent position, and a right-recumbent position.

In an Example 15, the method of any of Examples 10-14, wherein generating the study report comprises determining an impact on heart function resulting from the episode.

In an Example 16, a system for performing a sleep study, comprising: an implant able medical device (IMD) configured to be implanted within a patient's body, the IMD comprising a first sensor configured to obtain a first physiological parameter signal; a second sensor configured to obtain a second physiological parameter signal; and at least one processing device configured to: receive the first physiological parameter signal and the second physiological parameter signal; identify, based on a set of episode data, an occurrence of an episode during a sleep session, the episode corresponding to a sleep disorder, the set of episode data based on at least one of the first physiological parameter signal and the second physiological parameter signal; and generate, based on the first physiological parameter signal and the second physiological parameter signal, a study report, the study report comprising an indication of the episode.

In an Example 17, the system of Example 16, wherein the at least one processing device is further configured to identify, at a first time, an onset of the sleep session based on at least one of the first physiological parameter signal and the second physiological parameter signal.

In an Example 18, the system of Example 17, wherein the at least one processing device is further configured to identify, at a second time, an interruption of the sleep session based on at least one of the first physiological parameter signal and the second physiological parameter signal.

In an Example 19, the system of Example 16, wherein the first sensor comprises at least one of a respiration sensor, a heart rate sensor, an activity sensor, and a posture sensor.

In an Example 20, the system of Example 19, wherein the second sensor comprises at least one of a cardiac sensor, an activity sensor, a posture sensor, a heart sound sensor, an intrathoracic pressure sensor, and a blood pressure sensor.

In an Example 21, the system of Example 16, wherein the episode comprises at least one of a breathing cessation episode, an apnea episode, a hypopnea episode, and a restlessness episode.

In an Example 22 the system of Example 16, the study report comprising correlation information associated with the first physiological parameter signal and the second physiological parameter signal.

In an Example 23, the system of Example 22, wherein the correlation information comprises a correlation between a set of posture information and at least one of a set of apnea information, a set of restlessness information, a set of cardiac information, a set of heart sounds information, a set of blood pressure information, and a set of intrathoracic pressure information.

In an Example 24, the system of Example 16, wherein the at least one processing device is further configured to diagnose the sleep disorder based on a count of occurrences of the episode exceeding a threshold.

In an Example 25, a method of performing a sleep study associated with a subject, the method comprising: obtaining, using an implant able medical device (IMD), a first physiological parameter signal; obtaining a second physiological parameter signal; identifying, based on a set of episode data, an occurrence of an episode during a sleep session, the episode corresponding to a sleep disorder, the set of episode data based on at least one of the first physiological parameter signal and the second physiological parameter signal; and generating, based on the first physiological parameter signal and the second physiological parameter signal, a study report, the study report comprising an indication of the episode.

In an Example 26, the method of Example 25, further comprising: identifying, at a first time, an onset of the sleep session based on at least one of the first physiological parameter signal and the second physiological parameter signal; and identifying, at a second time, an interruption of the sleep session based on at least one of the first physiological parameter signal and the second physiological parameter signal.

In an Example 27, the method of Example 25, wherein generating the study report comprises: generating at least one waveform corresponding to the sleep session, the at least one waveform comprising at least one of a respiration waveform, an electrogram, a pressure waveform, a sound waveform, and an activity waveform; and annotating the at least one waveform to indicate the occurrence of the episode.

In an Example 28, the method of Example 25, wherein generating the study report comprises: identifying a plurality of occurrences of additional episodes, each of the plurality of occurrences of additional episodes comprising at least one of a breathing cessation episode, an apnea episode, a hypopnea episode, and a restlessness episode; determining a plurality of postures of the subject during the sleep session; correlating each of the plurality of occurrences of additional episodes with one of the plurality of postures of the subject; and generating correlation information associated with the correlated occurrences of additional episodes and postures.

In an Example 29, the method of Example 28, wherein the plurality of postures comprise at least one of a sitting position, a back-recumbent position, a front-recumbent position, a left-recumbent position, and a right-recumbent position.

In an Example 30, the method of Example 25, wherein generating the study report comprises determining an impact on heart function resulting from the episode.

In an Example 31, a method of performing a sleep study associated with a subject, the method comprising: obtaining, using a respiratory sensor implanted in the subject, a respiratory signal; obtaining, using a posture sensor implanted in the subject, a posture signal; obtaining, using a cardiac sensor, a cardiac signal, the cardiac signal comprising a heart rate signal; identifying, based on the posture signal and the heart rate signal, an onset of a sleep session; identifying, based on the respiratory signal, an occurrence of a respiratory episode during the sleep session; annotating the respiratory signal with an annotation corresponding to the respiratory episode; correlating, based on the posture signal, a posture with the respiratory episode; and generating a study report, the study report comprising an indication of the episode and correlation information indicating the correlated posture.

In an Example 32, the method of Example 31, further comprising: causing a display device to display a plurality of waveforms, each of the plurality of waveforms representing one of the respiration signal and the cardiac signal; and causing the display device to display a representation of the annotation.

In an Example 33, the method of Example 31, further comprising: determining an impact on heart function resulting from the respiratory episode; and including, in the study report, information associated with the determined impact.

In an Example 34, the method of Example 33, wherein determining the impact on heart function comprises: obtaining at least one of a heart sound signal, an electric cardiac signal, and a blood pressure signal; identifying a cardiac episode corresponding to the respiratory episode; and including, in the study report, correlation information associated with the respiratory episode and the corresponding cardiac episode.

In an Example 35, the method of Example 33, wherein determining the impact on heart function comprises: obtaining an activity signal; identifying a restlessness episode corresponding to the respiratory episode; including, in the study report, correlation information associated with the respiratory episode and the corresponding restlessness episode.

While the disclosed subject matter is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

As the terms are used herein with respect to ranges of measurements (such as those disclosed immediately above), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

Although the term “block” may be used herein to connote different elements illustratively employed, the term should not be interpreted as implying any requirement of, or particular order among or between, various steps disclosed herein unless and except when explicitly referring to the order of individual steps. Additionally, a “set” or “group” of items (e.g., inputs, algorithms, data values, etc.) may include one or more items, and, similarly, a subset or subgroup of items may include one or more items.

### BRIEF DESCRIPTION OF THE DRAWINGS

FIG. 1 shows an illustrative medical system, in accordance with certain embodiments of the present disclosure.

FIG. 2 shows a block diagram depicting an illustrative computing device, in accordance with certain embodiments of the present disclosure.

FIG. 3 shows a flow diagram depicting an illustrative method, in accordance with certain embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

Embodiments of the disclosure include systems and methods performing a sleep study, using an implant able medical device (IMD) in an environment other than that of a sleep study clinic (e.g., at the subject's home). In this manner, the information gathered may facilitate a more accurate understanding of the subject's sleep patterns, sleep disorders, and/or the like, because the information can be obtained in a natural (or otherwise typical) sleeping environment, as opposed to in a clinic, which typically involves a number of wires connected to the patient, and other atypical environmental factors.

In embodiments, data is obtained from an IMD by triggering a limited-time system behavior change. Embodiments include utilizing study prescriptions (e.g., sleep study prescriptions) that specify one or more criteria, procedures, parameters, and/or other aspects of obtaining the data. For example, study prescriptions may facilitate enabling sensor components, obtaining data, analyzing data, batching data obtained by an IMD, communicating the batched data to an external device, reconstructing the batched data at the external device, and/or the like. Study prescriptions may also include instructions for configuring one or more sensors, modifying one or more filters, modifying one or more sensor inputs (e.g. by changing a vector measured by a minute volume (MV) impedance component from focusing on changes in a lung to focusing on stroke volume of the heart), modifying one or more sensing parameters (e.g., sampling rate, sample storage rate, sensing thresholds, sensing durations), and/or the like.

Study prescriptions refer to sets of instructions, conditions, protocols, and/or the like, that specify one or more of an information gathering scheme, a communication scheme, an analysis scheme, and a reporting scheme, and may be configured, for example, to obtain information at a resolution sufficient for performing a certain analysis (e.g., associated with a diagnostic model), while managing the resulting impact to device longevity and/or performance. Examples of study prescriptions and implementation thereof may be found in U.S. Provisional Patent Application No. 62/276,383, to D. Ternes et al., filed on Jan. 8, 2016, the entirety of which is hereby incorporated herein by reference.

In this manner, for example, a particular sensor may be generally disabled (e.g., because it consumes relatively large amounts of power, is not necessary for a day-to-day or beat-to-beat operation), but may be able to be enabled in response to execution of instructions of a study prescription. For example, in embodiments, the filters of an accelerometer may be modified, according to a study prescription, to analyze data in different frequency ranges. In an implementation, for example, an IMD may be configured to generally use an accelerometer to drive rate and sample sensed measurements in a first frequency range, e.g., to facilitate rate-responsive pacing. A study prescription may be configured to cause the IMD to perform a sleep apnea study overnight and, accordingly, may cause the IMD to sample sensed measurements in a second frequency range so that the IMD can detect throat sounds. In this example, the study prescription may also be configured to disable the rate-responsive pacing functions during the night-time sleep study.

In embodiments, a clinician may determine a need or desire for obtaining data (information) from an IMD and may discuss this need for the data with the patient, after which the clinician may “prescribe” the data gathering study. The patient's implanted device may be set up to transmit data for prescribed period of time (e.g., automatically, via a wearable external monitoring device). The data may be, for example, transmitted in a continuous RF communication, batched, and/or the like. In embodiments, batching data may be dependent on a multitude of factors, e.g., the studies conducted, what channels are recording by default, whether any channels kick in after the first channel records something of interest for a study, and/or the like.

Embodiments may include any number of different considerations that may facilitate maximizing (or at least enhancing) data gathering while minimizing (or at least reducing) resulting impacts on the longevity of one or more components of the IMD. Such considerations may include, for example, storage capacity, power source depletion; and/or the like. Any number of various techniques may be implemented to facilitate these and/or other objectives. For example, rolling buffers may be implemented for managing the stored information. In embodiments, data may be overlapped to account for missed transmissions, such that when external devices piece the data back together (e.g., based on time stamps), the data may complete, and repeated data can be discarded. Data may also be synchronized with data from other sensors so that one parameter may be analyzed in the context of one or more other parameters. Any number of techniques for synchronizing data may be utilized including, for example, using sync signals as described in U.S. Provisional Patent Application No. 62/276,686, to P. Thakur et al., filed Jan. 8, 2016, the entirety of which is hereby incorporated by reference herein. In embodiments, the IMD may be configured to sense information at a lower resolution unless a trigger event (described in more detail below) is identified, at which time data may be gathered at a higher resolution. In embodiments, an external device may be passive and/or may actively request data from the IMD.

In embodiments, the number of times a study prescription can be enabled may be limited by the prescription, the IMD, the external device, and/or the like. In embodiments, a study prescription may also cause an IMD to provide information associated with the cumulative impact to the longevity of the IMD from implementing the study prescription, and may prompt a server or other device to obtain confirmation from a user before authorizing implementation of another study prescription, or another implementation of the same study prescription. Similarly, the IMD may perform a study prescription in stages, providing longevity impact information after the completion of each stage (with the system, perhaps, requiring a user confirmation to continue with the study prescription after each stage).

Additionally, to enhance efficiency, a study prescription may cause an IMD to turn off one or more channels when the IMD is gathering data. In embodiments, the study prescription may be configured to cause the IMD to first obtain the information that has the lowest longevity impact cost initially and then to dynamically determine how much additional information is needed, as captured information is analyzed. Embodiments may also facilitate dynamically switching frequencies at which communications are conducted, dynamically adjusting data sampling rates, data batching frequencies, and/or the like. Additionally, embodiments may facilitate remotely programming IMDs (e.g., to be remotely turned off, to remotely enable study prescriptions, to remotely adjust therapy, and/or the like).

According to embodiments, a sleep study may include identifying, monitoring, and analyzing sleep apnea, sleep posture, sleep interruptions, restlessness, restless leg syndrome, and any number of other factors that may impact the quality of sleep. In embodiments, a sleep study may be configured to provide a tossing and turning quantification, identify trips to the bathroom and/or other excursions, characterizing time to return to restful sleep after each interruption or excursion, detecting, identifying, and/or characterizing snoring, rasping, and/or rales, and/or the like. In embodiments, a sleep study prescription may be configured to identify specific factors leading to poor sleep quality and, thereby, assist a clinician and/or subject with diagnosis, therapy, and/or recommending sleep-improving actions.

In embodiments, a sleep study prescription may be implemented by one or more IMDs, EMDs, and/or other external devices. The sleep study prescription may be configured to facilitate an overnight sleep study, a sleep study that is performed over the course of a number of nights (e.g., two nights, three nights, each night for a week). In embodiments, the study prescription may be configured to cause an IMD and/or other device to periodically gather information and/or perform an in-depth apnea evaluation. For example, a longer-term sleep study may be performed in such a manner as to reduce longevity concerns (e.g., by increasing the amount of time between each data gathering/transmitting session, by reducing the number of occurrences of data gathering/transmission).

In embodiments, a study prescription may facilitate performing a sleep study over the course of multiple nights with one set-up procedure. That is, for example, upon enabling a study prescription, the one or more devices used for performing the sleep study may be configured to automatically (or in response to some condition) perform various tasks over a number of different nights. For example, an IMD may be configured to obtain a set of values of one or more parameters for certain amounts of time (or during certain time periods) each of the nights of the study. The IMD may additionally, or alternatively, be configured to obtain a set of values of one or more parameters each time a trigger event occurs during any of the nights of the study. Any number of other paradigms for obtaining information to be used in the sleep study may be configured in a study prescription. At the conclusion of the study, a report may be automatically generated and provided to a user (e.g., a clinician, a subject). The report may be generated by any device or combination of devices, and may be provided automatically and/or upon request, and may include, for example, information obtained during the sleep study, information derived from information obtained during the sleep study, recommendations for improving sleep, diagnostic information, and/or the like. In embodiments, the results of the sleep study may be used by one or more devices to dynamically adjust one or more types of therapy.

For example, embodiments may include identifying sleep apnea (and, in embodiments, determining the type and/or severity thereof) in ICD and CRT patients. Sleep apnea is a major co-morbidity in heart failure with a prevalence of about 52%. Yet, sleep apnea remains under-diagnosed, e.g., in ICD, CRT, and heart failure patients. Patient outcome may be adversely impacted by untreated sleep apnea, and sleep studies typically are performed in an artificial laboratory setting, which may not result in accurate reproductions of sleep events that occur in a natural sleep environment.

Embodiments of systems and/or methods described herein may be utilized to perform in-home sleep studies that may be performed over multiple nights in a more natural context, which may facilitate not only more accurate data, but also may be less expensive and inconvenient, as the patient may continue to go about their typical routine. For example, in-home sleep studies are more likely to capture impact of true medication compliance, alcohol use, and/or the like. Additionally, no external wires (or fewer), as compared to those of a sleep center, enable the patient to assume typical sleep positions (wires often end up tethering the patient into atypical sleep positions). Moreover, in-home sleep studies may be performed, according to embodiments, using IMDs already implanted in the subjects such as, for example, ICDs, CRT systems, and/or the like.

According to embodiments, an illustrative scenario may occur as follows. A patient presents with symptoms to a physician. Those symptoms may include, for example, shortness of breath, daytime sleepiness, and/or snoring. To determine whether the patient has Sleep Disordered Breathing (SDB), the physician may order an IMD-based sleep study (e.g., if the patient has an implanted pulse generator (IPG)). In embodiments, the IMD may already be programmed with instructions (the study prescription) for performing the study, while in other embodiments, those instructions may need to be provided to the IMD. In order to enable the study prescription, the physician may contact the IMD manufacturer or the study prescription provider to obtain a prescription code. By requiring a physician to obtain a prescription code, embodiments may facilitate any number of different billing arrangements, including one in which the physician may be reimbursed (by the provider of the IMD or study prescription).

The prescription code is entered into the IMD via an external device communicating with the IMD, which enables the study prescription. In embodiments, the prescription code may enable the IMD to perform a certain number of sleep studies (e.g., 5), and different codes may enable different numbers of sleep studies, different parameters associated with sleep studies, and/or the like. The study prescription may be configured to cause the IMD to obtain high resolution respiratory signals. Other sensors may be invoked, too, if necessary (e.g., if the prescription calls for the data, if a trigger event is identified, if analysis of the respiratory signals reveals characteristics about the data already collected that should be investigated using other types of data). Additional data collected by the IMD or an EMD may include, for example, posture and activity (e.g., to verify sleep and/or quantify disruptions of sleep such as sleep fragmentation, to correlate sleep characteristics to position); oxygen saturation (e.g., SpO2 obtained by external pulse oximeters); electroencephalogram (EEG) outputs; cyclic variation of heart rate (CVHR); Heart Rate Variability (HRV) associated with SDB; accelerometer readings to detect snoring and/or Obstructive Sleep Apnea (OSA). Other information that may be collected for a sleep study, e.g., by an IMD, may include EGM channels including marker and interval information, as determined by the IMD; respiration information, including identification and characterization of apneic events; posture information; and/or the like.

In embodiments, a sleep study may be configured to utilize activity sensors (e.g., accelerometers, microphones) to provide a tossing and turning quantification, identify trips to the bathroom and/or other excursions (which may be used, e.g., to rule out apnea and identify other causes of poor nights' sleep and day-time sleepiness such as, for example, prostrate issues, urinary tract infections and/or diabetes), and/or the like. Combined with HR, HRV, MV, and/or other sensor data, activity information may facilitate estimating sleep state and/or quality of sleep. Activity sensors may also, or alternatively, be used for detecting snoring, rasps, rales, and/or the like.

In embodiments, activity sensors may also be used for identifying and/or characterizing restless leg syndrome. For example, information may be obtained using an accelerometer in a rate response (RR) mode or a heart sounds mode. If using a RR mode, the ensemble average and sensor rectified average (SRA) may be removed, and the system may be configured to provide, for example, a waveform or other simple representation of the accelerometer readings over time. In an example, activity and/or posture sensors may be used to confirm that a patient is supine, and not active, but is otherwise experiencing periodic bursts of leg activity.

In embodiments, the collected data is provided to an EMD. The data may be “batched” to reduce RF power consumption. In embodiments, data from sleeping hours (e.g., which are detected based on time of day, accelerometer data, or both) may be streamed to an EMD. A physician or other user may review the collected data. In embodiments, the data may be provided to a sleep specialist (e.g., via the sleep specialist's user group), who may, for example, annotate sleep stage, annotate apneic and hypopneic events, based on activity data, respiratory data, and ECG data. Movements may be annotated for diagnosis of primary movement-related sleep disorders (RLS) or secondary symptoms of apnea, night terrors, and/or the like. In embodiments, sleep specialists may review annotated data. In embodiments, the system may invoke a component that automatically annotates and/or reviews the attached. Automatic annotation may be facilitated by user-provided criteria, machine-learning techniques, and/or the like. According to embodiments, a user (e.g., a physician) may be able to access the management server and register particular experts (e.g., experts for different clinical experts) so that the server may automatically provide the data to be reviewed.

A report may be generated from the study (manually and/or automatically) and reviewed for apnea diagnosis (e.g. by a sleep clinician). If evidence of disordered breathing is present, the physician may refer the patient for a full sleep study. In embodiments, data may be provided to the patient (e.g., via the patient's mobile device) that may describe the results of the study, provide recommendations for contacting the physician or changing behavior. Similarly, trigger event notifications may be pushed to a user device (e.g., a patient's mobile device, a clinician's workstation) to encourage an intervention such as, for example, performing a sleep study. In this manner, embodiments may facilitate avoiding unnecessary sleep studies; and may help in diagnose HF patients who need sleep therapy.

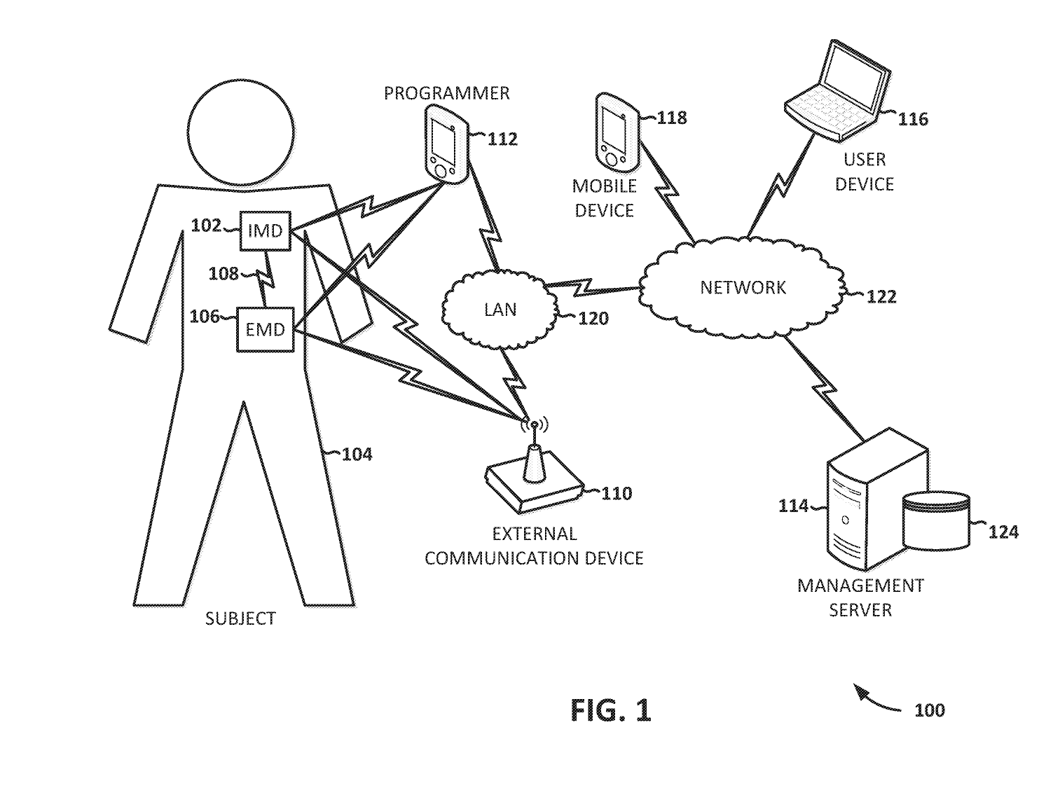

FIG. 1 shows an illustrative medical system 100, in accordance with embodiments of the disclosure. As shown in FIG. 1, the medical system 100 includes an IMD 102 configured to be implanted within the body of a subject 104, and an external monitoring device (EMD) 106, which is communicatively coupled to the IMD 102 via a communication link 108. In the illustrated embodiments, the medical system 100 is operatively coupled to the subject 104, and the IMD 102 is configured to communicate with the EMD 106 over the communication link 108. The subject 104 may be a human, a dog, a pig, and/or any other animal having physiological parameters that can be recorded. For example, in embodiments, the subject 104 may be a human patient.

In embodiments, the communication link 108 may be, or include, a wireless communication link such as, for example, a short-range radio link, such as Bluetooth, IEEE 802.11, a proprietary wireless protocol, and/or the like. In embodiments, for example, the communication link 108 may utilize Bluetooth Low Energy radio (Bluetooth 4.1), or a similar protocol, and may utilize an operating frequency in the range of 2.40 to 2.48 GHz. The term “communication link” may refer to an ability to communicate some type of information in at least one direction between at least two devices, and should not be understood to be limited to a direct, persistent, or otherwise limited communication channel. That is, according to embodiments, the communication link 108 may be a persistent communication link, an intermittent communication link, an ad-hoc communication link, and/or the like. The communication link 108 may refer to direct communications between the IMD 102 and the EMD 106, and/or indirect communications that travel between the IMD 102 and the EMD 106 via at least one other device (e.g., a repeater, router, hub, and/or the like). The communication link 108 may facilitate uni-directional and/or bi-directional communication between the IMD 102 and the EMD 106. For example, data and/or control signals may be transmitted between the IMD 102 and the EMD 106 to coordinate the functions of the IMD 102 and/or the EMD 106. In embodiments, patient data may be downloaded from one or more of the IMD 102 and the EMD 106 periodically or on command. The physician and/or the patient may communicate with the IMD 102 and the EMD 106, for example, to acquire patient data or to initiate, terminate and/or modify recording and/or therapy.

In embodiments, the IMD 102 and/or the EMD 106 may provide one or more of the following functions with respect to a patient: sensing, data analysis, and therapy. For example, in embodiments, the IMD 102 and/or the EMD 106 may be used to obtain any number of a variety of physiological, device, subjective, and/or environmental parameter signals associated with the subject 104, using electrical, mechanical, and/or chemical means. The IMD 102 and/or the EMD 106 may be configured to automatically gather data, gather data upon request (e.g., input provided by the subject, a clinician, another device, and/or the like), and/or any number of various combinations and/or modifications thereof. The IMD 102 and/or EMD 106 may be configured to store data related to the physiological, device, environmental, and/or subjective parameter signals and/or transmit the signals, and/or associated data, to any number of other devices in the system 100. In embodiments, the IMD 102 and/or the EMD 106 may be configured to analyze data and/or act upon the analyzed data. For example, the IMD 102 and/or EMD 106 may be configured to modify therapy, perform additional monitoring, generate reports, notify clinicians, and/or provide alarm indications based on the analysis of the data.

According to embodiments, the IMD 102 may include any type of IMD, any number of different components of an implant able system, and/or the like. For example, the IMD 102 may include a control device, a monitoring device, a pacemaker, an implant able cardioverter defibrillator (ICD), a cardiac resynchronization therapy (CRT) device and/or the like, and may be an implantable medical device known in the art or later developed, for providing therapy and/or diagnostic data about the subject 104 and/or the IMD 102. In various embodiments, the IMD 102 may include both defibrillation and pacing/CRT capabilities (e.g., a CRT-D device).

In embodiments, the IMD 102 may be implanted subcutaneously within an implantation location or pocket in the patient's chest or abdomen and may be configured to monitor (e.g., sense and/or record) physiological parameters associated with the patient's heart. In embodiments, the IMD 102 may be an implantable cardiac monitor (ICM) (e.g., an implantable diagnostic monitor (IDM), an implantable loop recorder (ILR)) configured to record physiological parameter signals such as, for example, one or more cardiac electrical signals, heart sounds signals, heart rate signals, blood pressure signals, oxygen saturation signals, and/or the like.

In embodiments, the IMD 102 may be configured to monitor physiological parameters that may include one or more signals indicative of a patient's physical activity level and/or metabolic level, such as an acceleration signal. In embodiments, the IMD 102 may be configured to sense intrathoracic impedance, from which various respiratory parameters may be derived, including, for example, respiratory tidal volume and minute ventilation. Sensors and associated circuitry may be incorporated in connection with the IMD 102 for detecting one or more body movement or body posture and/or position related signals. For example, accelerometers and/or GPS devices may be employed to detect patient activity, patient location, body orientation, and/or torso position. The IMD 102 may be configured to sense and/or record at regular intervals, continuously, and/or in response to a identified event. For example, in embodiments, the IMD 102 may be configured to identify a trigger event (described in more detail below) and communicate a notification of the trigger event to the EMD 106, which may perform one or more actions to enable a study prescription that may be implemented by the IMD 102 to acquire higher resolution data to confirm the trigger event, classify the trigger event, diagnose a related condition, and/or the like.

In various embodiments, the EMD 106 may be a device that is configured to be portable with the subject 104, e.g., by being integrated into a vest, belt, harness, sticker; placed into a pocket, a purse, or a backpack; carried in the subject's hand; and/or the like, or otherwise operatively (and/or physically) coupled to the subject 104. The EMD 106 may be configured to monitor (e.g., sense and/or record) physiological parameters associated with the subject 104 and/or provide therapy to the subject 104. For example, the EMD 106 may be, or include, a wearable cardiac defibrillator (WCD) such as a vest that includes one or more defibrillation electrodes. In embodiments, the EMD 106 may include any number of different therapy components such as, for example, a defibrillation component, a drug delivery component, a neurostimulation component, a neuromodulation component, a temperature regulation component, and/or the like. In embodiments, the EMD 106 may include limited functionality, e.g., defibrillation shock delivery and communication capabilities, with arrhythmia detection, classification and/or therapy command/control being performed by a separate device such as, for example, the IMD 102.

In embodiments, the EMD 106 may include sensing components such as, for example, one or more surface electrodes configured to obtain an electrocardiogram (ECG), one or more accelerometers configured to detect motion associated with the patient 104, one or more respiratory sensors configured to obtain respiration information, one or more environmental sensors configured to obtain information about the external environment (e.g., temperature, air quality, humidity, carbon monoxide level, oxygen level, barometric pressure, light intensity, sound, and/or the like) surrounding the patient 104, and/or the like. In embodiments, the EMD 106 may be configured to measure parameters relating to the human body, such as temperature (e.g., a thermometer), blood pressure (e.g., a sphygmomanometer), blood characteristics (e.g., glucose levels), body weight, physical strength, mental acuity, diet, heart characteristics, relative geographic position (e.g., a Global Positioning System (GPS)), and/or the like.

According to embodiments, the EMD 106 may be configured to measure subjective and/or perceptive data from the subject 104. Subjective data is information related to a patient's feelings, perceptions, and/or opinions, as opposed, for example, to objective physiological data. For example, EMD 106 may be configured to measure subject responses to inquiries such as “How do you feel?” and “How is your pain?” The EMD 106 may be configured to prompt the subject 104 and record subjective data from the subject 104 using visual and/or audible cues. In embodiments, the subject 104 can press coded response buttons or type an appropriate response on a keypad. In embodiments, subjective data may be collected by allowing the subject 104 to speak into a microphone and using speech recognition software to process the subjective data.

In embodiments, the EMD 106 may include a prescription enabler (discussed in further detail in U.S. Provisional Patent Application No. 62/276,383) that may be configured to automatically enable a study prescription when the EMD 106 is within communicating range of the IMD 102. In embodiments, enablement of the study prescription may require a password or other input, which may be received by the EMD 106. In other embodiments, the EMD 106 may include a button, switch, or other actuable mechanism that a patient or clinician may actuate to enable the study prescription. In other embodiments, the study prescription may be enabled at an earlier time (e.g., in the clinician's office, using a wand), but implemented later, in response to an input from the subject indicating, for example, that the subject is going to bed, having a certain feeling, and/or the like. In embodiments, the study prescription may be enabled within the IMD 102 earlier (e.g., in the clinician's office), and implemented only when the subject comes into proximity of the EDM 106 (or enabled within the IMD 102 and/or EMD 106 and implemented when the subject comes into proximity of another external device such as, for example, an external communication device 110).

As shown in FIG. 1, the system 100 includes the external communication device 110 and a programmer 112. In embodiments, the external communication device 110 and/or the programmer 112 may be, be similar to, include, or be included in, the EMD 106 and/or the mobile device 118, while in other embodiments, the external communication device 110 and/or the programmer 112 may be separate devices from the EMD 106. In embodiments, the external communication device 110 and/or the programmer 112 may be provided to the subject 104 and are often located within the subject's home.

According to embodiments, the external communication device 110 and/or the programmer 112 may be configured to send data to, and receive data from, a device, such as the IMD 102, the EMD 106, the other of the external communication device 110 and the programmer 112, and/or any number of other devices depicted or not depicted in FIG. 1. Such communications may be facilitated via communication links 108B-108I, any number of which may be, be identical to, be similar to, include, be coupled with, or be included within, the communication link 108A. The external communication device 110 and/or programmer 112 may operate as an interrogator of the IMD 102 and/or the EMD 106. In embodiments, the external communication device 110 and/or programmer 112 may perform one or more of the following functions: (1) data storage; (2) data analysis; (3) data forwarding; (4) patient interaction; (5) patient feedback; and (6) data communications. For example, the external communication device 110 and/or programmer 112 may facilitate communications between the devices 102 and 106 and a management server 114, a user device 116, a mobile device 118, and/or the like. The external communication device 110 and/or programmer 112 may, periodically or in real-time, interrogate and download into memory clinically relevant patient data. This data may include, for example, P and R-wave measurements, pacing, shocking events, lead impedances, pacing thresholds, battery voltage, capacitor charge times, ATR episodes with electrograms, tachycardia episodes with electrograms, histogram information, and/or any other clinical information necessary to ensure patient health and proper device function.

In embodiments, the external communication device 110 and/or programmer 112 may communicate with a network 120 that may be, for example, a local area network (LAN) in the subject's home or other location. The external communication device 110 and/or programmer 112 may be configured to systematically obtain information from the devices 102 and/or 106 while the patient is sleeping, for example. The obtained data may be transmitted through the network 120 and/or a network 122 to the management server 114. In addition, in embodiments the external communication device 110 and/or programmer 112 functions in a hybrid form, utilizing wireless communication when available and defaulting to a local wireless portal or a wired connection when the wireless communication becomes unavailable. In embodiments, the network 120 and the network 122 may be integrated within one another, may be the same network, and/or the like.

In embodiments, the external communication device 110 and/or programmer 112 may be in the form of a small device that is placed in an inconspicuous place within the subject's residence and may use radio frequency (RF) to communicate with the IMD 102 and/or EMD 106. The external communication device 110 and/or programmer 112 may be implemented as part of a commonly-used appliance in the subject's residence. For example, the external communication device 110 and/or programmer 112 may be integrated with an alarm clock that is positioned near the subject's bed. In another embodiment, the external communication device 110 and/or programmer 112 may be implemented as part of the subject's personal computer system. In another embodiment, the external communication device 110 and/or programmer 112 may include a hand-held device such as a PDA, cellular telephone, or other similar. The hand-held device may upload data to the management server 114 wirelessly. Additionally, or alternatively, the hand-held device may periodically be placed in a cradle or other similar device that is configured to transmit the data to the management server 114. In embodiments, the external communication device 110 and/or programmer 112 may perform analysis on data and provide immediate feedback, as well as perform a variety of self-diagnostic tests to verify that it is functioning properly and that communication with one or more other devices has not be compromised.

In embodiments of the system 100, one or more functions of the external communication device 110 and/or programmer 112 may be integrated into the IMD 102, the EMD 106, the user device 116, and/or the mobile device 118. In some embodiments, the devices may communicate directly with the management server 114, which may be located in the subject's home and/or at a remote location (e.g., the server 114 may be implemented, at least in part, as software having components instantiated by more than one device). The devices 102, 106, 110, and/or 112 may incorporate multi-mode wireless telecommunications such as cellular, BLUETOOTH, or IEEE 802.11B to communicate with the networks 120 and/or 122.

In embodiments, various devices of the system 100 may be configured to communicate during a given duty cycle. For example, the IMD 102, EMD 106, external communication device 110 and/or programmer 112 may be configured to communicate with the management server 114 (or other device) at given intervals, such as once a week. The IMD 102, EMD 106, external communication device 110 and/or programmer 112 may record data for the time period (e.g., a week) and transmit the data to the management server 114 (or other device) during the portion of the cycle that transmission is active and then conserve energy for the rest of the cycle. In another example, the IMD 102, EMD 106, external communication device 110 and/or programmer 112 conserve energy and only communicates with the management server 114 (or other device) when a trigger event or execution of a study prescription has occurred.

Various components depicted in FIG. 1 may operate together to form the monitoring system 100, which may be, for example, a computerized patient management and monitoring system. In embodiments, the system 100 may be designed to assist in monitoring the subject's condition, managing the subject's therapy, and/or the like. An illustrative patient management and monitoring system is the LATITUDE® patient management system from Boston Scientific Corporation, Natick Mass. Illustrative aspects of a patient management and monitoring system are described in ADVANCED PATIENT MANAGEMENT SYSTEM INCLUDING INTERROGATOR/TRANSCEIVER UNIT, U.S. Pat. No. 6,978,182 to Mazar et al., the entirety of which is hereby incorporated by reference herein.

Patient management and monitoring systems can provide large amounts of data about patients to users such as, for example, clinicians, patients, researchers, and/or the like. For example, such systems can store information about patient characteristics, patient sensor readings including electrocardiograms (EGMs), device settings, therapy deliveries, and/or the like. For example, in embodiments, medical devices such as the IMD 102 and/or the EMD 106 may obtain parameter values that include information associated with an apnea episode or other respiratory episode experienced by the patient. As it is used herein, the term “episode” refers to an occurrence of an event of interest (e.g., an abnormal event or event of other interest) or a potential occurrence of the event (e.g., where information appears to indicate an occurrence of the event). The term “episode” may also, or alternatively, refer to a time period during which an event of interest occurs or appears to occur. For example, an episode may refer to a cardiac episode (e.g., an arrhythmia, a cardiac pause), a sleep disturbance (e.g., an apnea episode, a hypopnea episode, a restlessness episode, a snoring episode), a psychological episode (e.g., a seizure or other epileptic episode), and/or the like. “Episode data” may include physiological parameter signals, and/or data derived therefrom and/or associated therewith, obtained before, during and/or after an episode, and may also include device settings, actions that were taken by the device, actions that were taken by a user, environmental parameters, and/or other information.

The episode data, or part of the episode data, corresponding to a particular episode may be analyzed using one or more adjudication algorithms to determine one or more classifications of the episode. For example, arrhythmia adjudication algorithms may be used to determine arrhythmia classifications and/or other types of characterizations about an arrhythmia episode; a sleep disturbance adjudication algorithm may be used to determine sleep disturbance classifications and/or other types of characterizations about a sleep disturbance episode; a psychological abnormality adjudication algorithm may be used to determine psychological abnormality classifications and/or other types of characterizations about a psychological episode; and/or the like.

In embodiments, medical devices such as the IMD 102 and/or the EMD 106 may obtain samples of interest such as, for example, parameter values that include information that may be, for example, stored and used as baseline information for comparing with captured parameter values for identifying episodes or performing other analyses. The samples of interest (e.g., baseline information) may be compared to episode data to facilitate classification and/or other characterization of episode data. In embodiments, baseline information may be used compared to other samples of interest from different measurement times, locations, sensors, and/or the like, to identify data trends, situations (e.g., certain stages of sleep, postures, etc.) in which episodes of certain types may be more likely to occur, and/or the like. In embodiments, baseline information may be dynamically captured. That is, for example, a sample of interest that is used as baseline information may be replaced periodically (e.g., according to a schedule) or in response to satisfaction of a baseline update criterion. For example, in response to identifying a certain trend in collected data, a certain number of episodes of a certain type, a persistent or recurring change in average parameter values, and/or any number of other types of observations, a medical device may be configured to capture a new sample of interest. In embodiments, the medical device may be configured to capture, based on a satisfaction of a criterion (or criteria), a different number of samples of interest, types of samples of interest, and/or the like. Additionally, or alternatively, the medical device may be configured to modify analyses performed using samples of interest (e.g., baseline information) in response to satisfaction of a criterion or criteria.

According to embodiments, an adjudication algorithm may be used to identify a particular event, referred to herein as a “trigger event,” that prompts further data gathering and analysis (e.g., further adjudications). For example, a medical device (e.g., the IMD 102 and/or the EMD 106) may obtain a first set of information, which may be analyzed to identify a trigger event. The trigger event may be, for example, a certain heart rate, EGM feature, snoring episode, apnea episode, and/or the like. In response to identifying the trigger event, the system may generate a study prescription that, when executed, facilitates enabling the IMD 102 to perform at least a portion of a study. As the term is used herein, a “study” is a monitoring activity that involves obtaining certain parameter values, storing certain parameter values, transmitting certain parameter values, and/or analyzing certain parameter values according to a study prescription, which includes one or more instructions, rules, schemes, and/or the like. For example, in embodiments, a study prescription may include a communication scheme that is configured based on IMD power consumption associated with information transmission from the IMD 102 to an EMD 106 or other device. In executing a study prescription, one or more components of the system 100 obtain and/or store a second set of information that may be analyzed using one or more adjudication algorithms to classify an episode, characterize the condition of a component of the IMD (e.g., a lead integrity), audit the effectiveness of a therapy regimen, and/or the like.

According to embodiments, classifications and/or characterization data can be stored in an adjudication database. In some examples, the characterization data may be sent to the medical device (e.g., IMD 102 and/or EMD 106) to be stored. Once a classification (e.g., an arrhythmia classification) has been generated for a particular episode or a group of episodes, it may be possible to provide patients and/or clinicians with many different types of reports related to the episode data. It may also be possible for the system to analyze the classifications and/or characterization data to provide programming recommendations for a medical device where certain conditions are present. It may also be possible to query the adjudication database for many different types of information that may be useful to clinicians, researchers, regulators, and/or the like.

In embodiments, episode adjudication for identifying a trigger event may be done by the IMD 102 and/or by the EMD 106. For example, a controller or controllers may be configured to extract certain features from a set of information that may include episode data, which may be useful in classifying an episode. The features may, in embodiments, be based on domain knowledge used by clinicians, engineers, technicians, and/or the like to classify the episode data. For example, in embodiments, an annotated respiration waveform and/or an electrogram may be used to determine if an apnea episode is associated with a certain posture, has a certain impact on cardiac function, and/or the like. In embodiments, episode adjudication for identifying a trigger event may be performed by any number of different components, and/or combinations of components, of the system 100.

According to embodiments, the management server 114 may be used to analyze information obtained in accordance with a study prescription. In embodiments, the management server 114 may additionally, or alternatively, be configured to identify a trigger event, generate a study prescription, provide reports to user devices 116 and/or mobile devices 118, manage patient information, configure therapy regimens, manage/update device software, and/or the like. In embodiments, the management server 114 may be, include, or be included within a server, a server cluster, a computer system, a cloud platform, an enterprise network, and/or the like. Additionally, although illustrated as a device, the management server 114 may, in embodiments, be implemented, at least in part, as software instantiated by any number of devices.

The management server 114 may, for example, index information using a database 124. The database 124 may be, or include, one or more tables, one or more relational databases, one or more multi-dimensional data cubes, one or more non-relational databases, and/or the like. Further, though illustrated as a single component, the database 124 may, in fact, be a plurality of databases 124 such as, for instance, a database cluster, which may be implemented on a single computing device or distributed among a number of computing devices, memory components, or the like.

The management server 114 may be configured to perform security functions, verification functions, and/or the like. Due to potential risks associated with inaccurate adjudication of episodes, identification of triggers, and adjustments in therapy provided by medical devices, it may be desirable for aspects of an at least partially automated system 100 to include safeguards such as, for example, verification of calculations, clinician oversight, and/or the like.

For example, before a study prescription is provided to the IMD 102, the management server 114 may provide a notification of the study prescription to a clinician or other user via the user device 116, mobile device 118, and/or the like. The user (e.g., clinician), in response to receiving the notification, may request a description of the study prescription. In embodiments, the notification of the study prescription may include a description thereof, and may include an indication of a longevity impact associated with the study prescription. As is explained in further detail below, a value may be determined that reflects an impact on the longevity of one or more components of the IMD 102 that is likely to result from execution of a particular study prescription. By presenting this longevity impact value to a user, along with a description of the study prescription, the user is provided with an opportunity to allow the study prescription to be executed or to prevent execution thereof, depending on whether the user believes that the impact on the longevity of the device is outweighed by the potential benefits of executing the study prescription. According to embodiments, the system 100 may include a component that performs this analysis in an automated fashion, based on criteria that may be provided by users and/or learned using a machine-learning technique.

The user (or component or automated process) may provide a confirmation (or denial) of the study prescription to the management server 114. In response to receiving the confirmation, the management server 114 may proceed with providing the study prescription to the IMD 102 for execution. In this manner, embodiments facilitate obtaining a confirmation of a study prescription, or aspects thereof, before implementing the study prescription. In embodiments, for example, a study prescription may be provided to the IMD 102 but may not be executable by the IMD 102 until the IMD 102 receives an enablement command from another device such as, for example, the EMD 106, the external communications device 110, the management server 114, and/or the like. The enablement command may be provided upon receiving a confirmation of the study prescription by, for example, a clinician. According to embodiments, the management server 114 may be configured to provide any number of other, or alternative, functions associated with patient management and/or monitoring.

In embodiments, the system 100 may be configured so that various components of the system 100 provide reporting to various individuals (e.g., patients and/or clinicians). For example, different levels of reporting may be provided by (1) the EMD 106 and/or the external communications device 110 and (2) the management server 114. The EMD 106 and/or the external communications device 110 may be configured to conduct preliminary analysis of data gathered from the IMD 102, and provide reporting should an acute situation (e.g., an episode such as a trigger event) be identified. For example, if the EMD 106 and/or the external communications device 110 detects that a significant heart arrhythmia is imminent or currently taking place, the EMD 106 and/or the external communications device 110 may provide reporting to the patient in the form of an audible or visual alarm.

In an example, upon identifying an apneic event or other sleep-related event, the IMD 102 may store episode data associated with the apneic event or other sleep-related event in a manner similar to a manner in which data associated with other types of identified events (e.g., tachycardia events, bradycardia events, etc.) are stored. That is, for example, upon identifying the apneic event or other sleep-related event, the IMD 102 may store data associated with some period of time before the occurrence of the episode, the time during the occurrence of the episode, and/or some period of time after the occurrence of the episode. This episode data may include, for example, cardiac data, respiratory data, activity data, posture data, and/or the like. In embodiments, the captured episode data, and/or data derived therefrom, may be sent to an external device for display, further analysis, and/or other processing. In embodiments, the IMD 102 may be configured to manage memory used for storing episode data and/or other parameters such as, for example, by utilizing a priority scheme that is configured to erase certain types of data when other types of data should be stored (e.g., by prioritizing the saving of data associated with tachycardia or other cardiac issues over data associated with sleep disorders). In this manner, embodiments may facilitate performing various aspects of sleep monitoring described herein using an IMD 102 even when an external device may not be available for receiving and/or processing data.

In another example, an alarm or alert may be triggered upon one of the monitored physiological parameters crossing a threshold. For example, in the case of monitoring and generating alerts for sleep apnea, a patient's Apnea-hypopnea Index (AHI), among other parameters, may be monitored and an alert generated when the AHI crosses a threshold a certain number of times or remains above a threshold for a certain period of time. The generated alert may initiate a prescription study or schedule a prescription study. The generated alert may be automatically sent to a clinician so that the clinician can further investigate the patient's sleep apnea.

In addition to forms of reporting including visual and/or audible information, the system 100 may also communicate with and/or reconfigure one or more of the devices 102, 106, 110, and/or 112. For example, if the IMD 102 is part of a cardiac rhythm management system, the management server 114 may communicate with the device 102 and reconfigure the therapy provided by the cardiac rhythm management system based on the data collected from one or more of the devices 102, 106, 110, and/or 112. In another embodiment, the management server 114 may provide to the EMD 106 and/or the external communications device 110 recorded data, an ideal range for the data, a conclusion based on the recorded data, and/or a recommended course of action. This information may be displayed on the EMD 106 and/or the external communications device 110 for the patient to review or made available for the patient and/or clinician to review.

Any number of various components of the system 100 depicted in FIG. 1 may be communicatively coupled via the networks 120 and/or 122. FIG. 1 illustrates one embodiment for the communication system 100. The networks 120 and/or 122 may be, or include, any number of different types of communication networks such as, for example, a bus network, a short messaging service (SMS), a local area network (LAN), a wireless LAN (WLAN), a wide area network (WAN), the Internet, a P2P network, custom-designed communication or messaging protocols, and/or the like. The networks 120 and/or 122 may include a combination of multiple networks.

A variety of communication methods and protocols may be used to facilitate communication between devices 102, 106, 110, 112, 114, 116, and/or 118. For example, wired and wireless communications methods may be used. Wired communication methods may include, for example and without limitation, traditional copper-line communications such as DSL, broadband technologies such as ISDN and cable modems, and fiber optics, while wireless communications may include cellular, satellite, radio frequency (RF), Infrared, etc.

For any given communication method, a multitude of standard and/or proprietary communication protocols may be used. For example and without limitation, protocols such as radio frequency pulse coding, spread spectrum, direct sequence, time-hopping, frequency hopping, SMTP, FTP, and TCP/IP may be used. Other proprietary methods and protocols may also be used. Further, a combination of two or more of the communication methods and protocols may also be used.

The various communications between the components of the system 100 may be made secure using several different techniques. For example, encryption and/or tunneling techniques may be used to protect data transmissions. Alternatively, a priority data exchange format and interface that are kept confidential may also be used. Authentication may be implemented using, for example, digital signatures based on a known key structure (e.g., PGP or RSA). Other physical security and authentication measures may also be used, such as security cards and biometric security apparatuses (e.g., retina scans, iris scans, fingerprint scans, vein scans, voice, facial geometry recognition, etc.). Conventional security methods such as firewalls may be used to protect information residing on one or more of the storage media of the advanced patient management system 100. Encryption, authentication and verification techniques may also be used to detect and correct data transmission errors.

In embodiments, varying levels of security may be applied to communications depending on the type of information being transmitted. For example, in embodiments, the management server 114 (or other device) may be configured to apply stricter security measures to confidential health care information than to demographic information. Similarly, even more security may be applied to communications of information used for controlling therapy, adjudicating episodes, and/or the like.

Additionally, in embodiments, communications among the various components of the system 100 may be enhanced using compression techniques to allow large amounts of data to be transmitted efficiently. For example, the devices 102, 106, 110, 112, 114, 116, and 118 may compress information prior to transmitting the information to another device. In embodiments, adaptive compression techniques may be employed such as, for example, the techniques disclosed in U.S. Pat. No. 8,849,682, the entirety of which is hereby incorporated by reference herein.

The illustrative patient management and monitoring system 100 shown in FIG. 1 is not intended to suggest any limitation as to the scope of use or functionality of embodiments of the present disclosure. Neither should the illustrative system 100 be interpreted as having any dependency or requirement related to any single component or combination of components illustrated therein. Additionally, various components depicted in FIG. 1 may be, in embodiments, integrated with various ones of the other components depicted therein (and/or components not illustrated), all of which are considered to be within the ambit of the present disclosure.

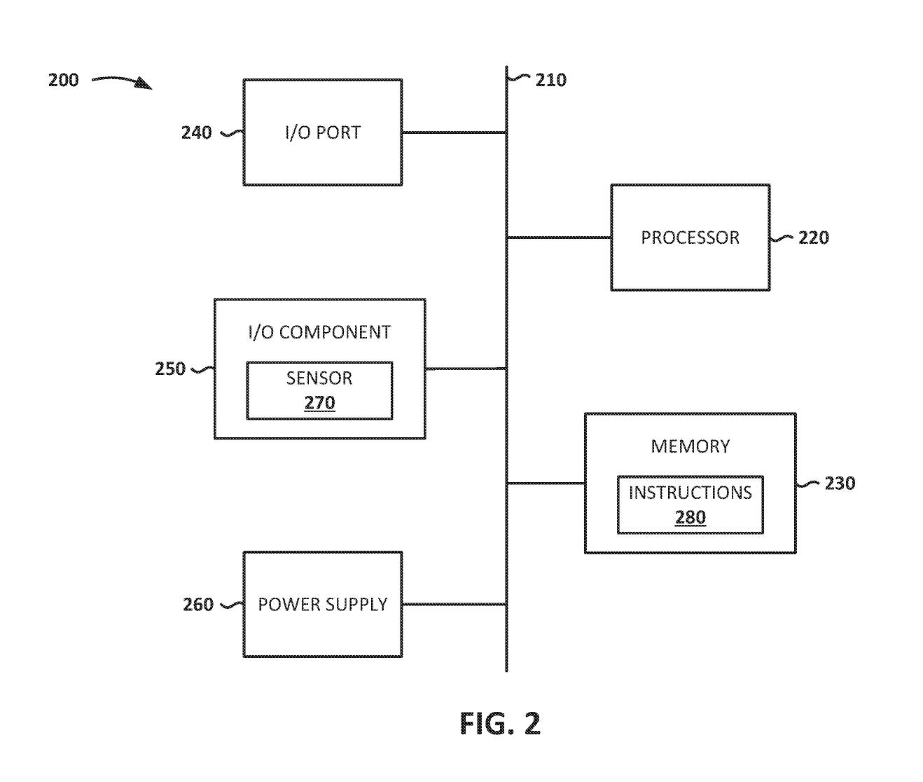

According to various embodiments of the disclosed subject matter, any number of the components depicted in FIG.1 (e.g., the IMD 102, the EMD 106, the external communication device 110, the programmer 112, the management server 114, the mobile device 116, and/or the user device 118) may be implemented on one or more computing devices. FIG. 2 is a block diagram depicting an illustrative computing device 200, in accordance with embodiments of the disclosure. The computing device 200 may include any type of computing device suitable for implementing aspects of embodiments of the disclosed subject matter. Examples of computing devices include specialized computing devices or general-purpose computing devices such “workstations,” “servers,” “laptops,” “desktops,” “tablet computers,” “hand-held devices,” “general-purpose graphics processing units (GPGPUs),” and the like, all of which are contemplated within the scope of FIGS. 1 and 2, with reference to various components of the system 100 and/or computing device 200.

In embodiments, the computing device 200 includes a bus 210 that, directly and/or indirectly, couples the following devices: a processor 220, a memory 230, an input/output (I/O) port 240, an I/O component 250, and a power supply 260. Any number of additional components, different components, and/or combinations of components may also be included in the computing device 200. The I/O component 250 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, a sensor 270, and/or the like.

The bus 210 represents what may be one or more busses (such as, for example, an address bus, data bus, or combination thereof). Similarly, in embodiments, the computing device 200 may include a number of processors 220, a number of memory components 230, a number of I/O ports 240, a number of I/O components 250, and/or a number of power supplies 260. Additionally any number of these components, or combinations thereof, may be distributed and/or duplicated across a number of computing devices.

In embodiments, the memory 230 includes computer-readable media in the form of volatile and/or nonvolatile memory and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In embodiments, the memory 230 stores computer-executable instructions 280 for causing the processor 220 to implement aspects of embodiments of system components discussed herein and/or to perform aspects of embodiments of methods and procedures discussed herein.

The computer-executable instructions 280 may include, for example, computer code, machine-useable instructions, and the like such as, for example, program components capable of being executed by one or more processors 220 associated with the computing device 200. Program components may be programmed using any number of different programming environments, including various languages, development kits, frameworks, and/or the like. Some or all of the functionality contemplated herein may also, or alternatively, be implemented in hardware and/or firmware.

The illustrative computing device 200 shown in FIG. 2 is not intended to suggest any limitation as to the scope of use or functionality of embodiments of the present disclosure. Neither should the illustrative computing device 200 be interpreted as having any dependency or requirement related to any single component or combination of components illustrated therein. Additionally, various components depicted in FIG. 2 may be, in embodiments, integrated with various ones of the other components depicted therein (and/or components not illustrated), all of which are considered to be within the ambit of the present disclosure.

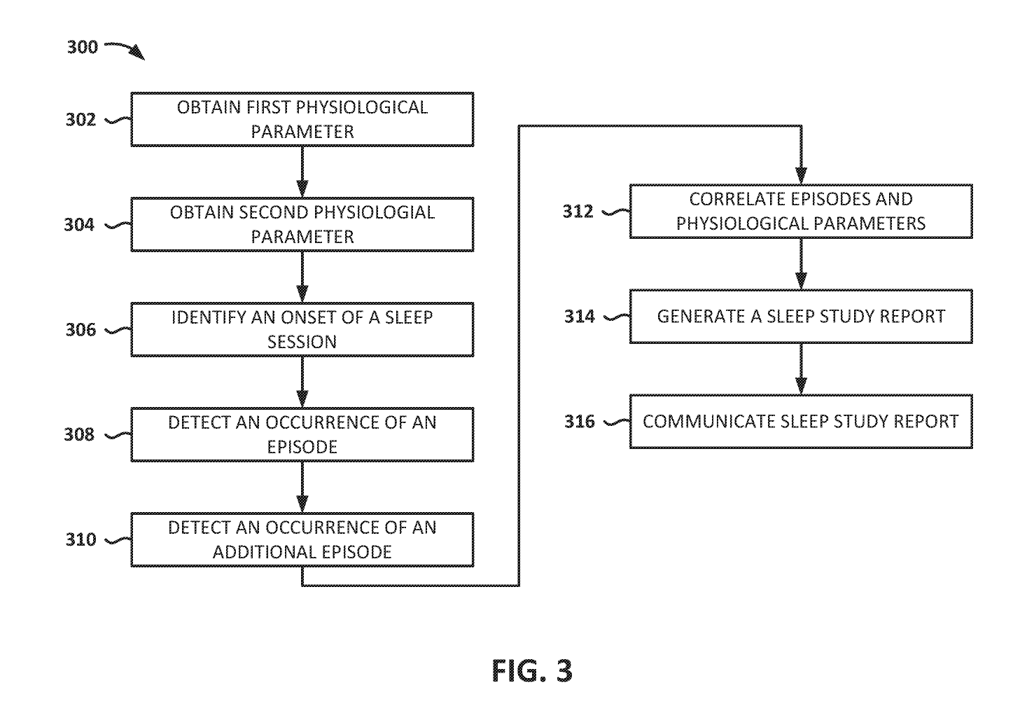

FIG. 3 is a flow diagram depicting an illustrative method 300 of generating a sleep study report. The functions and steps shown in the various blocks may be performed serially, in parallel, or in a different order as described below. Moreover, functions and steps may be carried out, individually or in combination, by the various devices described above.

Embodiments of the method 300 include obtaining at least first and second physiological parameters (blocks 302 and 304). The plurality of physiological parameters can include but are not limited to a patient's posture, movements, heart rate, intrathoracic pressure, oxygen saturation, EEG outputs, CVHR, HRV, respiratory information like those indicating breathing characteristics, heart sounds, and others mentioned above.

Embodiments of the method 300 include identifying an onset of a sleep session (block 306). Certain obtained physiological parameters can be used to determine an onset of sleep. For example, posture information may indicate a patient is lying down, and heart rate information may indicate that the patient's heart rate has dropped. Together, along with certain thresholds, (e.g., time a patient is recumbent, amount of decrease in patient's heart rate) these parameters may indicate an onset or end of a sleep session.

In some embodiments, physiological parameters may be obtained in response to identifying an onset of a sleep session (block 306). For example, it may be inefficient to repeatedly or continuously obtain certain physiological parameters outside of a sleep session. As such, an onset of a sleep session may initiate collection of certain physiological parameters. This feature is discussed in detail above with respect to initiating a prescription study. In some embodiments, an onset of sleep may initiate collection of higher resolution or more frequent collection of certain physiological parameters.

Embodiments of the method 300 include identifying one or more occurrences of an episode (blocks 308 and 310). In some embodiments, an IMD-based respiratory sensor can obtain information to identify episodes of breathing cessation. In some embodiments, intrathoracic pressure is obtained and used to differentiate between types of sleep apnea episodes. In some embodiments, a patient's activity is obtained and used to identify episodes of restlessness. For example, a restless episode may be determined to have occurred upon detecting non-periodic transient accelerometer signals without a change in posture. In some embodiments, a patient's posture is obtained and used to detect a patient's posture (e.g., supine, left recumbent, right recumbent, front recumbent, back recumbent)

Embodiments of the method 300 include correlating episodes and physiological parameters (block 312) for use to generate a sleep study report (block 314). In embodiments, correlation and generation involves summarizing a duration, count, and prevalence in various posture states of types of detected apneic episodes, restlessness episodes, and contiguous sleep episodes through one or more sleep sessions. For example, for each detected episode (e.g., apneic, restlessness), a generated report may correlate a patient's posture during the detected episode. For a detected apneic episode, a generated report may include IMD- and EMD-generated data (e.g., cardiac, respiratory, activity, posture) occurring before, during, and after the episode. The detected episodes may be scored, for example, as being a true apneic episode or a false apneic episode. In embodiments, a score may be calculated that indicates the likelihood of the detected event being a true and/or false apneic episode. A confidence level may be calculated to indicate a confidence of the score. Scoring of the detected episodes may be performed by an IMD before transmitting the scores to another device. In another example, a correlated and generated report may visually highlight a lack of apneic episodes along with prevalence of restlessness or times a patient stands up may indicate that the patient is not suffering from a sleep disorder but another disorder involving prostrate or urinary tract issues. In embodiments, correlation and generation involves summarizing cardiovascular interactions of apneic and restless episodes classified by posture state. For example, a report may highlight a percentage increase in S3 or S1/heart rate in a time period following an apneic or restlessness episode. A report may also highlight prevalence of premature ventricular contractions and associated heart rate turbulence in time periods preceding versus following apneic or restlessness episodes. In embodiments, correlation and generation involves synchronizing, in time, obtained physiological parameters so that time trends are indicated in a report. Reports may be annotated to highlight certain episodes, as mentioned above.

Embodiments of the method 300 include communicating the sleep study report (block 316). For example, if a physician initiates a prescription study, upon generation of the sleep study report, the report and underlying data may be communicated to the physician or a server for further analysis as discussed in detail above. In some embodiments, a sleep disorder can be automatically diagnosed upon detecting a number of episodes exceeding a predetermined threshold during a sleep session.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A system for performing a sleep study, comprising:
an implantable medical device (IMD) configured to be implanted within a patient's body, the IMD comprising a first sensor configured to obtain a first physiological parameter signal;
a second sensor configured to obtain a second physiological parameter signal; and
at least one processing device configured to:
receive the first physiological parameter signal and the second physiological parameter signal;
identify, based on a set of episode data, an occurrence of an episode during a sleep session, the episode corresponding to a sleep disorder, the set of episode data based on at least one of the first physiological parameter signal and the second physiological parameter signal; and
generate, based on the first physiological parameter signal and the second physiological parameter signal, a study report, the study report comprising an indication of the episode.

2. The system of claim 1, wherein the at least one processing device is further configured to identify, at a first time, an onset of the sleep session based on at least one of the first physiological parameter signal and the second physiological parameter signal.

3. The system of claim 2, wherein the at least one processing device is further configured to identify, at a second time, an interruption of the sleep session based on at least one of the first physiological parameter signal and the second physiological parameter signal.

4. The system of claim 1, wherein the first sensor comprises at least one of a respiration sensor, a heart rate sensor, an activity sensor, and a posture sensor.

5. The system of claim 4, wherein the second sensor comprises at least one of a cardiac sensor, an activity sensor, a posture sensor, a heart sound sensor, an intrathoracic pressure sensor, and a blood pressure sensor.

6. The system of claim 1, wherein the episode comprises at least one of a breathing cessation episode, an apnea episode, a hypopnea episode, and a restlessness episode.

7. The system of claim 1, the study report comprising correlation information associated with the first physiological parameter signal and the second physiological parameter signal.

8. The system of claim 7, wherein the correlation information comprises a correlation between a set of posture information and at least one of a set of apnea information, a set of restlessness information, a set of cardiac information, a set of heart sounds information, a set of blood pressure information, and a set of intrathoracic pressure information.

9. The system of claim 1, wherein the at least one processing device is further configured to diagnose the sleep disorder based on a count of occurrences of the episode exceeding a threshold.

10. A method of performing a sleep study associated with a subject, the method comprising:
obtaining, using an implantable medical device (IMD), a first physiological parameter signal;
obtaining a second physiological parameter signal;
identifying, based on a set of episode data, an occurrence of an episode during a sleep session, the episode corresponding to a sleep disorder, the set of episode data based on at least one of the first physiological parameter signal and the second physiological parameter signal; and
generating, based on the first physiological parameter signal and the second physiological parameter signal, a study report, the study report comprising an indication of the episode.

11. The method of claim 10, further comprising:
identifying, at a first time, an onset of the sleep session based on at least one of the first physiological parameter signal and the second physiological parameter signal; and
identifying, at a second time, an interruption of the sleep session based on at least one of the first physiological parameter signal and the second physiological parameter signal.

12. The method of claim 10, wherein generating the study report comprises:
generating at least one waveform corresponding to the sleep session, the at least one waveform comprising at least one of a respiration waveform, an electrogram, a pressure waveform, a sound waveform, and an activity waveform; and
annotating the at least one waveform to indicate the occurrence of the episode.

13. The method of claim 10, wherein generating the study report comprises:
identifying a plurality of occurrences of additional episodes, each of the plurality of occurrences of additional episodes comprising at least one of a breathing cessation episode, an apnea episode, a hypopnea episode, and a restlessness episode;
determining a plurality of postures of the subject during the sleep session;
correlating each of the plurality of occurrences of additional episodes with one of the plurality of postures of the subject; and
generating correlation information associated with the correlated occurrences of additional episodes and postures.

14. The method of claim 13, wherein the plurality of postures comprise at least one of a sitting position, a back-recumbent position, a front-recumbent position, a left-recumbent position, and a right-recumbent position.

15. The method of claim 10, wherein generating the study report comprises determining an impact on heart function resulting from the episode.

16. A method of performing a sleep study associated with a subject, the method comprising:
obtaining, using a respiratory sensor implanted in the subject, a respiratory signal;
obtaining, using a posture sensor implanted in the subject, a posture signal;
obtaining, using a cardiac sensor, a cardiac signal, the cardiac signal comprising a heart rate signal;
identifying, based on the posture signal and the heart rate signal, an onset of a sleep session;
identifying, based on the respiratory signal, an occurrence of a respiratory episode during the sleep session;
annotating the respiratory signal with an annotation corresponding to the respiratory episode;
correlating, based on the posture signal, a posture with the respiratory episode; and
generating a study report, the study report comprising an indication of the episode and correlation information indicating the correlated posture.

17. The method of claim 16, further comprising:
causing a display device to display a plurality of waveforms, each of the plurality of waveforms representing one of the respiration signal and the cardiac signal; and
causing the display device to display a representation of the annotation.

18. The method of claim 16, further comprising:
determining an impact on heart function resulting from the respiratory episode; and
including, in the study report, information associated with the determined impact.

19. The method of claim 18, wherein determining the impact on heart function comprises:
obtaining at least one of a heart sound signal, an electric cardiac signal, and a blood pressure signal;
identifying a cardiac episode corresponding to the respiratory episode; and
including, in the study report, correlation information associated with the respiratory episode and the corresponding cardiac episode.

20. The method of claim 19, wherein determining the impact on heart function comprises:
obtaining an activity signal;
identifying a restlessness episode corresponding to the respiratory episode;
including, in the study report, correlation information associated with the respiratory episode and the corresponding restlessness episode.
